(19) United States
(12) Patent Application Publication
(10) Pub. No.: US 2008/0300304 A1
(43) Pub. Date: Dec. 4, 2008

(54) Ginger Fraction For Inhibiting Human Cyp Enzymes

(76) Inventors: Thomas Ebner, Ingelheim (DE); Eva Christine Ludwig-Schwellinger, Ummendorf (DE); Stefan Blech, Warthausen (DE); Rolf-Stefan Brickl, Warthausen (DE)

Correspondence Address: MICHAEL P. MORRIS;BOEHRINGER INGELHEIM USA CORPORATION, 900 RIDGEBURY RD, P. O. BOX 3686, RIDGEFIELD, CT, 06877-0368, US

(21) Appl. No.: 12/158,374

(22) PCT Filed: Dec. 20, 2006
(86) PCT No.: PCT/EP2006/069975

§ 371 Date: Jun. 20, 2008

(30) Foreign Application Priority Data
Dec. 22, 2005 (DE) 102005062144.9

(51) Int. Cl. A61K 31/225; C07C 43/23; A61K 31/085; C07C 49/00; A61K 31/12; C07C 69/003

(52) U.S. Cl. 514/548; 568/652; 514/719; 568/308; 514/678; 560/194; 435/184

(57) ABSTRACT

The present invention relates to a process for preparing a ginger fraction, the fraction prepared by this process and the use thereof on its own or combined with drugs for inhibiting human cytochrome P450 (CYP) enzymes (particularly cytochrome P450 3A4, CYP3A4) for positively influencing the oral bioavailability and pharmacokinetics of active substances.

The present invention relates to a process for preparing a ginger fraction, the fraction prepared by this process and the use thereof on its own or combined with drugs for inhibiting human cytochrome P450 (CYP) enzymes (particularly cytochrome P450 3A4, CYP3A4) for positively influencing the oral bioavailability and pharmacokinetics of active substances.

### BACKGROUND TO THE INVENTION

Cytochrome P450 (CYP) enzymes play a central part in drug metabolism. They are found primarily in the liver but also in the intestinal wall, lungs, kidneys and other extrahepatic organs. Orally administered active substances may demonstrate poor bioavailability as a result of the so-called “first-pass effect”, for example those active substances which are subject to metabolisation in the intestinal wall or liver before reaching the systemic circulation.

If the first-pass metabolism is inhibited, a significant increase in the bioavailability of orally administered active substances can be achieved (Gibbs, Megan A. and Hosea, Natalie A.: Factors affecting the clinical development of cytochrome P450 3A substrates; Clin. Pharmacokinet. 2003; 42(11), 969-984). Many examples of active substance-active substance interactions which result in a bioavailability higher than that of the active substance administered are based on such effects. In such cases the first-pass metabolism of the active substance is inhibited by another active substance administered simultaneously.

Inhibiting the first-pass metabolism may, in addition to increasing the bioavailability of an active substance, significantly reduce the variability in bioavailability, which is known to increase as absolute bioavailability decreases. By reducing the variability in bioavailability the therapeutic success of an oral drug therapy is critically improved, as there is a lower incidence of exposure to excessively high drug levels (risk of unwanted side effects) or excessively low drug levels (risk of therapeutic failure).

Such effects may have certain advantages in drug therapy. For example the HIV drug lopinavir has inadequate bioavailability because of the first-pass metabolism by CYP3A4. If it is administered in a fixed-dose combination with ritonavir, which is a potent inhibitor of CYP3A4, a significantly higher oral bioavailability is achieved for lopinavir.

However, there are only limited possibilities of combining an active substance with poor oral bioavailability with another active substance or substance resembling an active substance in order to reduce the first-pass effect. This is due mainly to the mode of activity of the additional active substance. Thus, for example, too low a bioavailability of a cardiovascular drug cannot be increased by simultaneously giving an anti-retroviral active substance (indicated for HIV infection) to non-HIV-infected patients for ethical reasons. Even permitted active substances are not licensed for the purpose of inhibiting enzymes that metabolise active substances.

Instead, drug additives may be useful in this respect. For example, some constituents of grapefruit juice are potent inhibitors of CYP3A4 and drug transporters in the intestinal wall. The prior art contains numerous examples demonstrating that taking the drug together with grapefruit juice has dramatic effects on the pharmacokinetics, safety and efficacy of orally administered active substances, such as e.g. simvastatin, cyclosporin A, terfenadine etc. (Ameer, Barbara and Weintraub, Randy A.: Drug interactions with grapefruit juice; Clin. Pharmacokinet. 1997; 33(2):103-121).

### PRIOR ART

Ginger (Zingiber officinalis) is a traditional food ingredient in many parts of the world and is also used as a phytopharmaceutical for various applications. For example, powdered ginger root is available as a preparation for preventing seasickness.

It contains about 5 to 8% of a viscous liquid balsam (oleoresin), which contains a non-steam-volatile peppery or hot fraction as well as a volatile ethereal oil fraction. The pale yellow ethereal oil makes up about 20 to 25% of the oleoresin. The composition of the ethereal oil is subject to considerable fluctuations depending on its origin. It contains as its main ingredient sesquiterpene hydrocarbons of the bisabolone type, particularly (−)-α-zingiberene and also (−)-β-sesquiphellandrene, (−)-β-bisabolene, (+)-ar-curcumene and acyclic α-farnesene (Deutsche Apothekerzeitung 1997, 137(47), 40-46).

The main component of the hot fraction, making up about 25% of the oleoresin, constitutes the homologous series of the gingerols (HagerROM 2002: Zingiberis rhizoma, Springer Verlag, Heidelberg).

### DETAILED DESCRIPTION OF THE INVENTION

Surprisingly, in vitro tests on the inhibition of CYP by various active substances and other compounds have shown that potent inhibition of various human CYPs may be achieved by means of a ginger fraction obtained by an extraction process according to the invention.

This fraction shows a higher inhibitory potency (IC₅₀ in the range below 1 μg/ml) both compared to the commercially available total ginger extract (the so-called oleoresin) and also compared to the highly volatile fraction of ethereal ginger oil (IC₅₀ approx. 23 μg/ml), which is separated off in the first extraction step.

The fraction obtained here is poorly soluble in hexane and differs in this characteristic from the fraction of the ethereal oil, which has already been shown to inhibit CYP3A4 (U.S. Pat. No. 5,665,386).

The process according to the invention starts from a commercially obtainable oleoresin and comprises a number of extraction steps using organic and aqueous solvents.

A first object of the present invention is thus a process for isolating a ginger fraction while separating off the ethereal oil, comprising the steps of
(a) extracting an oleoresin with a non-polar organic solvent;
(b) extracting the combined residues from step (a) with warm water and discarding the supernatant.

The residues thus obtained have an IC₅₀ value of 30.5 to 42.0 μg/mL for CYP3A4. This value is achieved with human liver microsomes in the experiment described in the experimental section.

In a preferred embodiment the residue thus obtained is further purified by a process comprising the steps of
(c) extracting the combined residues from step (b) with warm alcohol and
(d) concentrating the combined supernatants from step (c).

The fraction obtained in step (d) may be dissolved in an alcohol, preferably methanol or ethanol, and optionally further fractionated, for example by solid phase extraction and stepwise elution.

Non-polar organic solvents which may be used in step (a) include according to the invention low-boiling alkane solvents such as, for example, hexane, heptane, octane, pentane or cyclohexane, petrochemical distillates, propellants and solvents such as for example petrol, kerosene, petroleum ether, petroleum and other low-boiling, volatile and non-polar solvents such as for example diethyl ether, tert.-butyl-methylether, tetrahydrofuran, benzene, toluene and xylenes, while hexane is preferably used.

The alcohol used in steps (c) and (e) may be selected from among methanol, ethanol, isopropanol, n-propanol, n-butanol and other positionally isomeric butanols, n-pentanol and other positionally isomeric pentanols and may be identical or different. Preferably, methanol is used. The extraction agent in each case is used in amounts of from 4 to 10 mL/g, preferably 4 to 7 mL/g, of the oleoresin used.

The aqueous extractions are preferably carried out at a temperature of from 50 to 80° C., particularly preferably 65 to 75° C.

As an alternative to this method extractions may also be carried out with suitable aqueous organic acids or, instead of liquid-liquid extraction with organic solvents, solid phase extractions with suitable non-polar absorbents may also be carried out.

The extractions carried out in steps (a), (b) and (c) may be carried out once or several times, and the phases containing the desired product from the various extractions of one step may be combined. Preferably the extraction is carried out three times in each step and the phases containing the product are combined. The combined phases are then further processed.

A second object of the present invention is the ginger fraction according to the invention, which may be obtained by one of the processes according to the invention.

A ginger fraction which contains at least one compound of general formulae

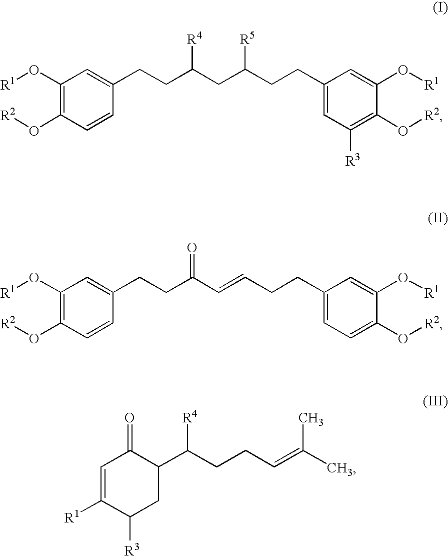

wherein
R¹ denotes H, CH₃,
R² denotes H, CH₃,
R³ denotes H, OH, OCH₃,
R⁴ denotes H, O, OH, OCH₃, OC(O)CH₃ and
R⁵ denotes H, O, OH, OCH₃, OC(O)CH₃,
   one of the enantiomers or diastereomers thereof, is preferred.

Examples of particularly preferred compounds of the previously mentioned general formula I to VI include:

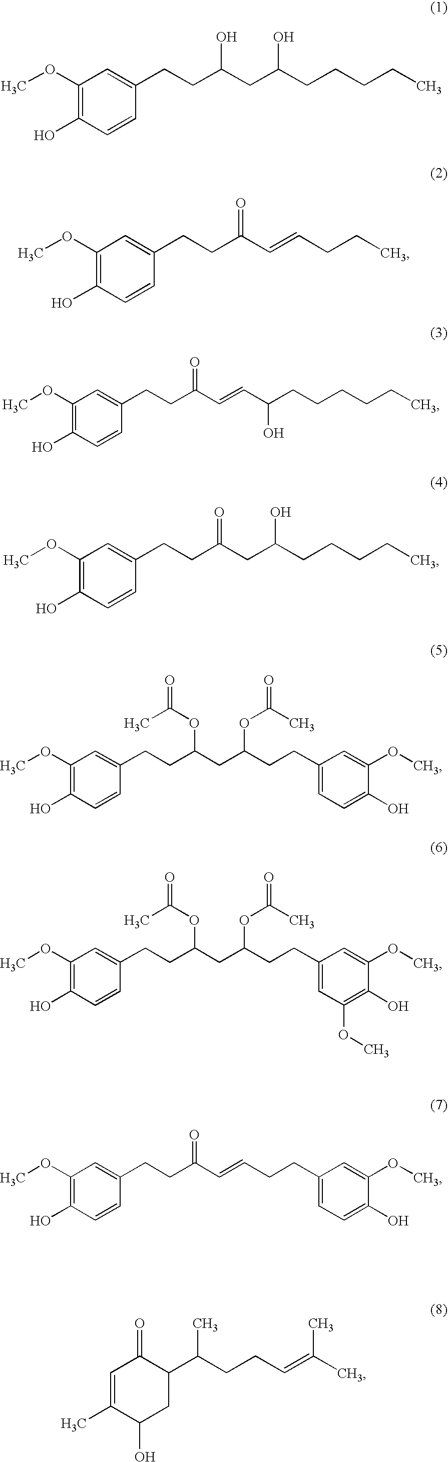

the enantiomers and the diastereomers thereof.

The compounds of general formulae I to III were identified from the ginger fraction obtained according to the invention. In order to characterise this ginger fraction more precisely and establish its contents, it was suitably further purified with the aim of isolating purified fractions of individual ingredients.

In order to do this, the ginger fraction obtained according to the invention was further purified by solid phase extraction on a C18 phase. The eluant of the solid phase extraction was dried out and investigated further by semipreparative high pressure liquid chromatography (HPLC). This was done by injecting fairly small aliquots of 5 to 10 mg into the semipreparative HPLC system. The eluant of the HPLC column was then collected in 60 to 65 individual fractions and each of the fractions thus obtained was investigated for its inhibitory effect on various P450 test reactions. The results of these investigations showed clearly defined zones (peaks) of higher inhibitory potency.

To clarify the chemical structure of the constituents of the individual fractions, selected samples were further purified and concentrated by repeated HPLC and then investigated by mass spectrometry and NMR spectroscopy.

Compounds (1) to (8) identified according to the invention are the typical ingredients of the non-volatile hot fraction of ginger which have already been sufficiently described in the literature. In addition to various modification products of gingerol and the various homologues thereof, a known main ingredient of ginger, [6]-gingerol (4), was also found.

This confirmed that the ginger fraction prepared by the process described is derived from the non-volatile hot fraction and the inhibition of the CYP enzymes is brought about by ingredients of the gingerol type and the structural modifications and breakdown products thereof.

A third object of the present invention is the use of the ginger fraction according to the invention and one or more of the compounds of general formulae I to III isolated therefrom for preparing a pharmaceutical composition for inhibiting cytochrome P450 enzymes, particularly cytochrome P450 3A4, 1A2, 2C19 and 2C9.

Preferably, also, the cytochromes P450 1A2, P450 2C19 and P450 2C9 are inhibited.

A fourth object of the present invention is the use of the ginger fraction according to the invention as well as one or more of the compounds of general formulae I to III isolated therefrom, in conjunction with a pharmaceutical composition for preparing a pharmaceutical composition for inhibiting human cytochrome P450 (CYP) enzymes, particularly cytochrome P450 3A4, 1A2, 2C19 and 2C9, for positively influencing the oral bioavailability and pharmacokinetics of active substances.

Preferably also, the cytochromes P450 1A2, 2C19 and 2C9 are inhibited.

Many active substances have low oral bioavailability, caused by the so-called first-pass metabolism. This is the metabolic breakdown of orally administered active substances in the small intestine or in the liver, even before they are able to travel through the bloodstream to their target organ.

The active substances mentioned previously, i.e. the pharmacologically active constituents of drugs, may be selected from among the drugs for acting upon the cardiovascular system in its widest sense, including those substances which serve to influence the composition of the blood (e.g. blood lipids); drugs acting on the central nervous system; drugs for treating metabolic disorders (e.g. diabetes mellitus); drugs for treating inflammatory processes in the widest sense; drugs for influencing the immune system; drugs for treating infections by bacteria, protozoa, multi-cellular parasites, viruses, fungi or prions; drugs for stopping or alleviating degenerative processes in various organs, particularly the brain, and drugs for treating cancer.

By the term “drugs” are meant substances and preparations of substances which are intended, by administration to or in the human or animal body,
1. to cure, alleviate, prevent or detect diseases, ailments, physical injury or pathological disorders;
2. to show up the nature, state or functions of the body or mental states;
3. to replace active substances or bodily fluids produced by the human or animal body;
4. to ward off, eliminate or render harmless pathogens, parasites or substances alien to the body or
5. to influence the nature, state or functions of the body or mental states.

Cytochrome P450 (CYP) enzymes in this case are enzymes from the family of the cytochrome P450 monooxygenases which are involved in the metabolism of drugs according to current scientific knowledge. In particular they are all P450 enzymes of the families CYP1A, CYP1B, CYP2A, CYP2B, CYP2C, CYP2D, CYP2E, CYP2F, CYP2J, CYP3A, CYP4A.

A fifth object of the present invention relates to a process for preparing a pharmaceutical composition for increasing the bioavailability of a pharmaceutical compound for oral administration, comprising orally administering the pharmaceutical compound together with a ginger fraction according to one of claims 8 to 10 to a person requiring such treatment, the ginger fraction being administered in an amount which is needed to increase the bioavailability of the pharmaceutical compound as compared with administration of the pharmaceutical compound on its own.

The pharmaceutical compound is characterised in that it is metabolised by cytochrome P450 enzymes, preferably by P450 3A4, 1A2, 2C9 and 2C19.

A sixth object of the present invention relates to a pharmaceutical formulation containing the ginger fraction which may be obtained according to the invention or at least one compound of general formulae I to III, the enantiomers or diastereomers thereof, optionally together with one or more pharmaceutically acceptable carriers and/or diluents for improving the oral bioavailability and pharmacokinetics of active substances.

Preferably a pharmaceutical composition of this kind consists of two or more components which are optionally physically separate from one another and comprises:
(a) a first component consisting of the ginger fraction according to the invention and one or more pharmaceutically acceptable diluents and/or carriers; and
(b) a second component containing a pharmaceutical composition, comprising a pharmaceutical compound which is metabolised by cytochrome P450 enzymes, and one or more pharmaceutically acceptable diluents and/or carriers.

In a preferred pharmaceutical composition the first component consists of at least one compound of general formulae I to III, the enantiomers or diastereomers thereof.

In a more preferred pharmaceutical composition the first component consists of at least one compound of formulae (1) to (8), the enantiomers or diastereomers thereof.

The pharmaceutical composition contained in the second component is preferably metabolised by the enzymes cytochrome P450 1A2, 3A4, 2C9 and 2C19.

As pharmaceutically acceptable carriers and/or diluents, maize starch, lactose, glucose, microcrystalline cellulose, magnesium stearate, polyvinylpyrrolidone, citric acid, tartaric acid, water, water/ethanol, water/glycerol, water/sorbitol, water/polyethyleneglycol, propyleneglycol, cetylstearylalcohol, carboxymethylcellulose or fatty substances such as hard fat or suitable mixtures thereof may be incorporated in the usual way into conventional galenic preparations such as tablets, coated tablets, capsules, powders, suspensions, solutions, metered dose aerosols or suppositories.

### EXPERIMENTAL SECTION

10 g of an oleoresin (Eramex Aromatics GmbH) are extracted three times with 50 mL hexane and the supernatant (organic phase) is discarded. The residues are combined and extracted three times with 40 mL of water heated to 70° C. The supernatant is discarded again and the combined residues are extracted three times with 40 mL of methanol heated to 70° C. The residue is discarded. The supernatant obtained is concentrated by rotary evaporation and dissolved in methanol again. Diagram 1 shows an overview of the extraction process of the ginger fraction according to the invention of the oleoresin with separation of the ethereal oils.

The ginger fraction thus obtained was then purified further by solid phase extraction on a C18 phase. The eluant of the solid phase extraction was dried and investigated further by semipreparative high pressure liquid chromatography (HPLC). For this, small aliquots of 5 to 10 mg were injected into the semipreparative HPLC system. The eluant of the HPLC column was then collected in 60 to 65 individual fractions and each of the fractions thus obtained was investigated for its inhibitory effect on various P450 test reactions. The results of these investigations (FIGS. 2 to 5) showed clearly demarcated zones (peaks) of higher inhibitory potency.

Experiments with Human Liver Microsomes

### Test (A):

### Erythromycin N-Demethylation is Used as a Specific Test Reaction for CYP 3A4

The ginger fraction or the various fractions of the extraction process are investigated for their inhibition of CYP. This involves incubating 100 μg of human liver microsomes with 0.01 to 100 μg of ginger fraction and 7.34 μg (10 nmol) of erythromycin at pH 7.4 in the presence of NADPH. The inhibition of the CYP activity is determined by comparison with control incubations with no ginger extract (with the same concentration of solvent).

### Test B:

### Phenacetin O-Deethylation is Used as a Specific Test Reaction for CYP 1A2.

The ginger fraction or the various fractions of the extraction process are investigated for their inhibition of CYP. This involves incubating 125 mg of human liver microsomes with 0.01 to 100 mg ginger fraction and 5 nmol phenacetin at pH 7.4 in the presence of NADPH (1 mM) in a total volume of 250 μl. The inhibition of the CYP activity is determined by comparison with control incubations with no ginger extract (with the same concentration of solvent).

### Test C:

### S-Mephenytoin 4′-Hydroxylation is Used as a Specific Test Reaction for CYP 2C19.

The ginger fraction or the various fractions of the extraction process are investigated for their inhibition of CYP. This involves incubating 125 mg of human liver microsomes with 0.01 to 100 mg ginger fraction and 12.5 nmol S-mephenytoin at pH 7.4 in the presence of NADPH (1 mM) in a total volume of 250 μl. The inhibition of the CYP activity is determined by comparison with control incubations with no ginger extract (with the same concentration of solvent).

### Test D:

### Tolbutamide Hydroxylation is Used as a Specific Test Reaction for CYP 2C9.

The ginger fraction or the various fractions of the extraction process are investigated for their inhibition of CYP. This involves incubating 125 mg of human liver microsomes with 0.01 to 100 mg ginger fraction and 37.5 nmol tolbutamide at pH 7.4 in the presence of NADPH (1 mM) in a total volume of 250 μl. The inhibition of the CYP activity is determined by comparison with control incubations with no ginger extract (with the same concentration of solvent).

### Formulations

The prerequisite for the inhibition of CYP 3A4 is that the ginger extract is largely dissolved in the intestinal tract. As the ginger extract is very poorly soluble in water, it was necessary to develop formulations that dissolved well in aqueous media. For this reason, formulation screening was carried out with a variety of pharmaceutically conventional excipients. More extensive tests were then carried out with particularly suitable excipients to obtain formulations which were optimum in terms of both quality and quantity.

1. Selection of Functional Excipients with a High Supersaturation of Ginger Extracts in Aqueous Media
1.1 Preparation of Formulations with Meltable Excipients

30 mg ginger extract were heated, with stirring, with 60 mg of the excipient in an aluminium plate with round depressions to a temperature which was about 5° C. above the melting temperature of the excipient, and mixed with thorough stirring. Then the mixture was cooled with stirring until a solid preparation was obtained. This was then used directly for in vitro testing.

1.2 Preparation of Formulations with Non-Meltable Excipients

200 mg ginger extract were dissolved in 2 ml alcohol, preferably ethanol. Then 300 μl in each case were intensively mixed with 60 mg of the excipient, and then the alcohol was evaporated off. The solid form obtained was then used directly for in vitro testing.

### 1.3 Results of the In Vitro Releases

### Method:

30 mg formulation, which contained 10 mg ginger extract in each case, were stirred into 20 ml of water at a temperature of 37° C. and the release was determined after 2, 5, 10, 15, 20, 25 and 30 minutes by UV measurement at 280 and 358 nm. This active substance charge corresponds to a human dose of 100 mg, which is taken together with 200 ml of water. Unformulated ginger extract was used as a reference substance. The releases were calculated from the quotient of the extinctions of the respective formulations and the extinctions of the reference form.

Table 1 provides an overview of the most important results.

TABLE 1
Overview of maximum releases of various
formulations in water at 37° C.

| | | supersaturation | |
|---|---|---|---|
| excipient | release [%] | factor | suitability |
| without | 4.5 | 1.0 | |
| tartaric acid | 2.4 | 0.5 | no |
| Cutina HR | 3.0 | 0.7 | no |
| Gelucire 50/13 | 6.0 | 1.3 | no |
| Stearylalcohol | 6.0 | 1.3 | no |
| Precirol ATO5 | 7.0 | 1.6 | no |
| Suppocire AM | 13.0 | 2.9 | conditional |
| Gelucire 39/01 | 13.0 | 2.9 | conditional |
| Gelucire 43/01 | 13.0 | 2.9 | conditional |
| Labrafil M1944 | 15.0 | 3.3 | conditional |
| Meglumin | 25.3 | 5.6 | good |
| PEG 6000 | 54.0 | 12.0 | good |
| Cremophor EL | 83.0 | 18.4 | good |
| Poloxamer 188 | 98.0 | 21.8 | good |
| Gelucire 44/14 | 100.0 | 22.2 | good |

It is apparent that even at the selected high active substance load of 33% with the most suitable formulations the ginger extract was released completely, corresponding to a more than 20-fold supersaturation. Good resorption of the ginger extract is thus ensured

2. Formulation Examples for Combinations of Ginger Extract and Active Substances the Bioavailability of which can be Increased Using Ginger Extract

In each case combinations of the ginger extract according to the invention and a pharmaceutical substance whose bioavailability is to be increased are used. This may be done using various formulation approaches:
(i) Ginger extract and active substance are formulated separately and then administered simultaneously or with a short interval between taking the ginger formulation and the formulation of the pharmaceutical composition (in order to achieve the saturation of Cyp 3A4 beforehand).
(ii) Semi-finished products of the ginger extract and active substance are prepared, which are then further processed to make a final fixed combination, the release of the ginger extract and medicament being matched to one another. This can be done, for example, by compressing a granulated ginger preparation and granulated pharmaceutical composition with tabletting excipients to produce a tablet, or multiparticulate formulations of ginger extract and medicament are together packed into a capsule.
(iii) Ginger extract and medicament are formulated together as a fixed medicament combination.

Some examples of the various types of formulation will now be described, which provide an illustration of the scope of the invention without limiting the overall scope.

### Example 1

### Preparation of a Ginger Formulation by Embedding the Ginger Extract in a Melt of Gelucire 44/14 and Packing into Hard Capsules

20 g ginger extract and 40 g Gelucire 44/14 are stirred at 50° C. until a homogeneous melt is formed. Using a liquid fill apparatus 75 mg of the melt obtained is transferred with stirring into a hard #5 capsule (gelatine or HPLMC). This corresponds to a dosage of 25 mg ginger extract. Analogously, 150 mg of melt (corresponding to a dosage of 50 mg ginger extract) may be packed into a #4 capsule, 225 mg of melt (corresponding to a dosage of 75 mg ginger extract) into a #2 capsule, 300 mg of melt (corresponding to a dosage of 100 mg ginger extract) into a #2 el capsule or 450 mg of melt (corresponding to a dosage of 150 mg ginger extract) into a #0 capsule.

### Example 2

### Preparation of a Ginger Formulation by Embedding the Ginger Extract in a Melt of Poloxamer 188 and Transferring into Hard Capsules

20 g ginger extract and 40 g Poloxamer 188 are stirred at 64° C. until a homogeneous melt is formed. Using a liquid fill apparatus 75 mg of the melt obtained are transferred into a hard capsule #5 (gelatine or HPLMC) with stirring. This corresponds to a dosage of 25 mg ginger extract. Analogously 150 mg of melt (corresponding to a dosage of 50 mg ginger extract) may be transferred into a #4 capsule, 225 mg of melt (corresponding to a dosage of 75 mg ginger extract) into a #2 capsule, 300 mg of melt (corresponding to a dosage of 100 mg ginger extract) into a #2 el capsule or 450 mg of melt (corresponding to a dosage of 150 mg ginger extract) into a #0 capsule.

### Example 3

### Preparation of a Ginger Formulation by Embedding the Ginger Extract in a Melt in Cremophor EL and Transferring into Hard Capsules

20 g ginger extract, 40 g Cremophor EL and 15 g microcrystalline cellulose are stirred at 48° C. until a homogeneous melt is formed in which the microcrystalline cellulose is uniformly suspended. Using a liquid fill apparatus 93.8 mg of the melt obtained are transferred with stirring into a #5 hard capsule (gelatine or HPLMC). This corresponds to a dosage of 25 mg ginger extract. Analogously 187.5 mg of melt (corresponding to a dosage of 50 mg ginger extract) may be transferred into a #4 capsule, 281.3 mg of melt (corresponding to a dosage of 75 mg ginger extract) into a #1 capsule, 375 mg of melt (corresponding to a dosage of 100 mg ginger extract) into a #1 capsule or 562.5 mg of melt (corresponding to a dosage of 150 mg ginger extract) into a #0 capsule.

### Example 4

### Preparation of a Ginger Formulation by Embedding the Ginger Extract in a Melt in PEG 6000 and Transferring into Hard Capsules

20 g ginger extract and 40 g PEG 6000 are stirred at 67° C. until a homogeneous melt is formed. Using a liquid fill apparatus 75 mg of the melt obtained is transferred with stirring into a hard capsule #5 (gelatine or HPLMC). This corresponds to a dosage of 25 mg ginger extract. Analogously 150 mg of melt (corresponding to a dosage of 50 mg ginger extract) may be packed into a #4 capsule, 225 mg of melt (corresponding to a dosage of 75 mg ginger extract) into a #2 capsule, 300 mg of melt (corresponding to a dosage of 100 mg ginger extract) into a #2 el capsule or 450 mg of melt (corresponding to a dosage of 150 mg ginger extract) into a #0 capsule.

### Example 5

### Preparation of a Ginger Formulation by Embedding the Ginger Extract in a Melt in PEG 6000/Poloxamer 188 and Transferring into Hard Capsules

20 g ginger extract, 20 g Poloxamer 188 and 20 g PEG 6000 are stirred at 67° C. until a homogeneous melt is formed. Using a liquid fill apparatus 75 mg of the melt obtained are packed with stirring into a hard capsule #5 (gelatine or HPLMC). This corresponds to a dosage of 25 mg ginger extract. Analogously 150 mg of melt (corresponding to a dosage of 50 mg ginger extract) may be packed into a #4 capsule, 225 mg of melt (corresponding to a dosage of 75 mg ginger extract) into a #2 capsule, 300 mg of melt (corresponding to a dosage of 100 mg ginger extract) into a #2 el capsule or 450 mg of melt (corresponding to a dosage of 150 mg ginger extract) into a #0 capsule.

### Example 6

### Preparation of a Ginger Formulation by Extrusion of the Ginger Extract with Poloxamer 188 and Transferring into Hard Capsules

20 g ginger extract and 40 g Poloxamer 188 are mixed dry and at 52° C. extruded in a 16 mm twin-screw extruder with a 1 mm die plate and head removal. The roughly 1 mm long cylinders formed are rounded off in a spheronizer at about 51° C. and then using a capsule filling machine packed into hard capsules (gelatine or HPLMC). If 75 mg are packed into a #5 capsule, this corresponds to a dosage of 25 mg ginger extract. Analogously 150 mg extrudate (corresponding to a dosage of 50 mg ginger extract) may be packed into a #4 capsule, 225 mg extrudate (corresponding to a dosage of 75 mg ginger extract) into a #2 capsule, 300 mg extrudate (corresponding to a dosage of 100 mg ginger extract) into a #2 el capsule or 450 mg extrudate (corresponding to a dosage of 150 mg ginger extract) into a #0 capsule.

### Example 7

### Preparation of a Ginger Formulation by Extrusion of the Ginger Extract with PEG 6000 and Transferring into Hard Capsules

20 g ginger extract and 40 g PEG 6000 are mixed dry and at 55° C. extruded in a 16 mm twin-screw extruder with a 1 mm die plate and head removal. The roughly 1 mm long cylinders formed are rounded off in a spheronizer at about 51° C. and then using a capsule filling machine packed into hard capsules (gelatine or HPLMC). If 75 mg are packed into a #5 capsule, this corresponds to a dosage of 25 mg ginger extract. Analogously 150 mg extrudate (corresponding to a dosage of 50 mg ginger extract) may be packed into a #4 capsule, 225 mg extrudate (corresponding to a dosage of 75 mg ginger extract) into a #2 capsule, 300 mg extrudate (corresponding to a dosage of 100 mg ginger extract) into a #2 el capsule or 450 mg extrudate (corresponding to a dosage of 150 mg ginger extract) into a #0 capsule.

### Example 8

### Preparation of a Ginger Formulation by Extrusion of the Ginger Extract with Poloxamer 188/PEG 6000 and Transferring into Hard Capsules

20 g ginger extract, 20 g Poloxamer 188 and 20 g PEG 6000 188 are mixed dry and at 53° C. extruded in a 16 mm twin-screw extruder with a 1 mm die plate and head removal. The roughly 1 mm long cylinders formed are rounded off in a spheronizer at about 51° C. and then using a capsule filling machine packed into hard capsules (gelatine or HPLMC). If 75 mg are packed into a #5 capsule, this corresponds to a dosage of 25 mg ginger extract. Analogously 150 mg extrudate (corresponding to a dosage of 50 mg ginger extract) may be packed into a #4 capsule, 225 mg extrudate (corresponding to a dosage of 75 mg ginger extract) into a #2 capsule, 300 mg extrudate (corresponding to a dosage of 100 mg ginger extract) into a #2 el capsule or 450 mg extrudate (corresponding to a dosage of 150 mg ginger extract) into a #0 capsule.

### Example 9

### Preparation of a Ginger Formulation by Extrusion of the Ginger Extract with Poloxamer 188/PEG 6000 and Transferring into Hard Capsules

20 g ginger extract, 40 g Cremophor EL and 20 g microcrystalline cellulose are intensively mixed and at 50° C. extruded in a 16 mm twin-screw extruder with a 1 mm die plate and head removal. The roughly 1 mm long cylinders formed are rounded off in a spheronizer at about 51° C. and then using a capsule filling machine packed into hard capsules (gelatine or HPLMC). If 100 mg are packed into a #4 capsule, this corresponds to a dosage of 25 mg ginger extract. Analogously 200 mg extrudate (corresponding to a dosage of 50 mg ginger extract) may be packed into a #3 capsule, 300 mg extrudate (corresponding to a dosage of 75 mg ginger extract) into a #2 el capsule, 400 mg extrudate (corresponding to a dosage of 100 mg ginger extract) into a #1 el capsule or 600 mg extrudate (corresponding to a dosage of 150 mg ginger extract) into a #0 el capsule.

The capsules obtained according to Examples 1 to 9 are used as free combinations with standard commercial formulations of active substances, the availability of which is reduced by CYP3A4 enzymes.

TABLE 2
Examples of active substances which
are substrates for CYP3A4 enzymes

| active substance | oral availability [%] |
|---|---|
| simvastatin | 4 |
| lovastatin | 4 |
| saquinavir | 4 |
| docetaxel | 8 |
| atorvastatin | 12 |
| felodipine | 15 |
| sirolimus | 15 |
| vardenafil | 15 |
| tacrolimus | 16 |
| verapamil | 17 |
| rifabutin | 20 |
| valspodar | 34 |
| cisapride | 38 |
| sildenafil | 38 |
| lopinavir | low |

The two medicament forms are taken either simultaneously or with a time delay of about 15 minutes between taking the ginger formulation and the active substance of reduced availability; taking the active substance after a time delay is preferable as it ensures that the CYP3A4 systems are saturated.

3. Pellet Formulations for Active Substances which are Substrates for CYP3A4

### Example 10

### Preparation of a Simvastatin Formulation by Wet Extrusion of Simvastatin with Microcrystalline Cellulose

20 g simvastatin, 2 g citric acid and 18 g microcrystalline cellulose are mixed dry and extruded at ambient temperature and with the simultaneous addition of water in a 16 mm twin-screw extruder with a 0.8 mm die plate. The extruded strips produced are broken up and rounded off in a spheronizer at ambient temperature and then dried.

### Example 11

### Preparation of a Lovastatin-Formulation by Wet Extrusion of Lovastatin with Microcrystalline Cellulose

20 g lovastatin and 30 g microcrystalline cellulose are mixed dry and at ambient temperature and extruded at ambient temperature and with the simultaneous addition of water in a 16 mm twin-screw extruder with a 0.8 mm die plate. The extruded strips produced are broken up and rounded off in a spheronizer at ambient temperature and then dried.

### Example 12

### Preparation of a Verapamil-Formulation by Wet Extrusion of Verapamil with Microcrystalline Cellulose

75 g verapamil and 25 g microcrystalline cellulose are mixed dry and extruded at ambient temperature and with the simultaneous addition of water in a 16 mm twin-screw extruder with a 0.8 mm die plate. The extruded strips produced are broken up and rounded off in a spheronizer at ambient temperature and then dried.

All the active substances of Tables 2 and 3 may be formulated analogously, the ratio of active substance: microcrystalline cellulose being selected so that the entire formulation is in the range from 100 to 300 mg, i.e. with low doses of active substances (<=40 mg active substance) 40 to 80% cellulose are used, while with higher doses of active substances only the minimum amount of >=20% cellulose needed for good workability is used. If desired other excipients may also be added to the formulations, for improving solubility and/or for stabilisation.

### Example 13

### Fixed Pharmaceutical Combinations of Non-Delayed Release Active Substance Pellets of CYP3A4 Substrates and Ginger Formulations in Capsules

By weighing out the respective pellet formulations in amounts which correspond to the desired dosages in each case, it is very easy to achieve all the desired combinations.

Some Examples are given in Table 3.

TABLE 3
Examples of different active substance combinations

| | | fill | | | |
|---|---|---|---|---|---|
| | | amounts of | ginger | | fill amounts |
| active substance | dose of | pellets of | formulation | | of pellets of |
| formulation | active | active | example; | | ginger |
| according to | substance | substance | dose of | | formulation |
| Example 11 | [mg] | [mg] | ginger [mg] | | [mg] |
| Simvastatin | 20 | 40 | 2 | 25 | 75 |
| Example 10 | | | | | |
| Simvastatin | 80 | 160 | 2 | 100 | 300 |
| Example 10 | | | | | |
| Lovastatin | 40 | 100 | 2 | 75 | 225 |
| Example 11 | | | | | |
| Verapamil | 80 | 106.4 | 2 | 100 | 300 |
| Example 12 | | | | | |

### Example 14

### Pellet Formulations with Delayed Release of Active Substances, which are Substrates for CYP3A4

100 g pellets of Examples 10 to 12 or analogous pellets of examples of other active substances are coated with a retardant coating of the following composition in a Hüttlin-Coater Microlab or other suitable apparatus:

| | | |
|---|---|---|
| hydroxypropylmethylcellulose-phthalate | 2.25 | g |
| Eudragit S | 0.25 | g |
| talc | 0.5 | g |
| triethylcitrate | 0.5 | g |
| isopropanol | 50 | ml |

After drying, pellets are obtained from which the active substance is only released after a lag time of about 15 minutes. Combining them with non-delayed release pellets ensures that CYP3A4 is already inhibited when the active substance is flooding in.

### Example 15

### Fixed Pharmaceutical Combinations of Delayed Release Active Substance Pellets of CYP3A4 Substrates and Ginger Formulations in Capsules

By weighing out the respective pellet formulations in amounts which correspond to the desired dosages in each case, it is very easy to achieve all the desired combinations.

Some Examples are given in Table 4.

TABLE 4
Examples of different active substance combinations

| formulation of | | fill amount | ginger | | |
|---|---|---|---|---|---|
| active | dose of | of pellets | formulation | | fill amount |
| substance | active | of active | example; | | of pellets of |
| according | substance | substance | dose of | | ginger |
| to Example 11 | [mg] | [mg] | ginger | | formulation |
| Simvastatin | 20 | 41.3 | 2 | 25 | 75 |
| Example 10 | | | | | |
| Simvastatin | 80 | 165 | 2 | 100 | 300 |
| Example 10 | | | | | |
| Lovastatin | 40 | 103.5 | 2 | 75 | 225 |
| Example 11 | | | | | |
| Verapamil | 80 | 109.7 | 2 | 100 | 300 |
| Example 12 | | | | | |

### Example 16

### Fixed Pharmaceutical Combinations of (Non)Delayed-Release Active Substance Pellets of CYP3A4 Substrates and Ginger Formulations in Tablets

The ginger-containing pellets in Examples 6 to 9 and the active substance-containing pellets in Examples 10 to 12 can also be compressed into tablets with excipients suitable for tablet-making:

Thus, for example, 40 g of pellets from Example 10 and 75 g pellets from Example 4 are mixed with 120 g microcrystalline cellulose, 40 g lactose and 7.5 g AcDiSol and 2.5 g magnesium stearate and compressed into tablets with a total weight of 285 mg, containing 20 mg simvastatin and 25 mg ginger extract.

Many other combinations of Examples 6 to 9 and 10 to 12 may be prepared analogously.

### Example 17

### Fixed Pharmaceutical Combinations of Non-Delayed-Release Active Substance Pellets of CYP3A4 Substrates and Ginger Formulations in Tablets

After extrusion the ginger-containing pellets of Examples 6 to 9 are ground up using suitable mills, e.g. centrifugal mills, to form granules with particle sizes in the range from 100 to 500 μm, and similarly the active substance-containing extrudates of Examples 10 to 12, which are ground up after drying. The granules can be compressed with excipients suitable for tabletting.

Thus, for example, 40 g of ground granules from Example 10, 75 g ground granules from Example 4 are mixed with 80 g microcrystalline cellulose, 20 g Lactose and 5 g AcDiSol and 1.5 g magnesium stearate and compressed into tablets with a total weight of 219.5 mg, containing 20 mg simvastatin and 25 mg ginger extract. Many other combinations of Examples 6 to 9 and 10 to 12 may be prepared analogously.

### BRIEF DESCRIPTION OF THE FIGURES

FIG. 1 shows an overview of the process for extracting the ginger fraction according to the invention of an oleoresin while separating off the ethereal oils.

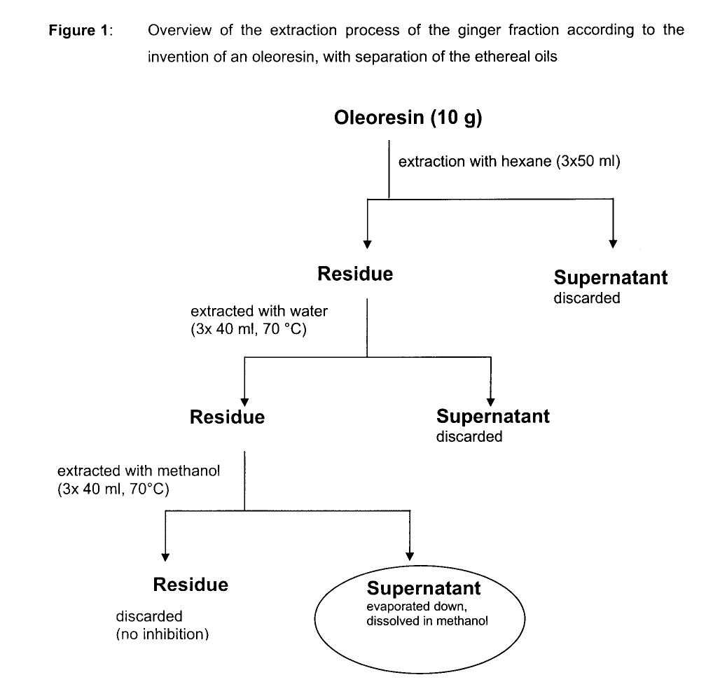

FIG. 2 shows the HPLC separation of a ginger extract and the measurement of the inhibitory potency relative to CYP3A4 for the eluted HPLC fractions (collecting period: 1 minute).

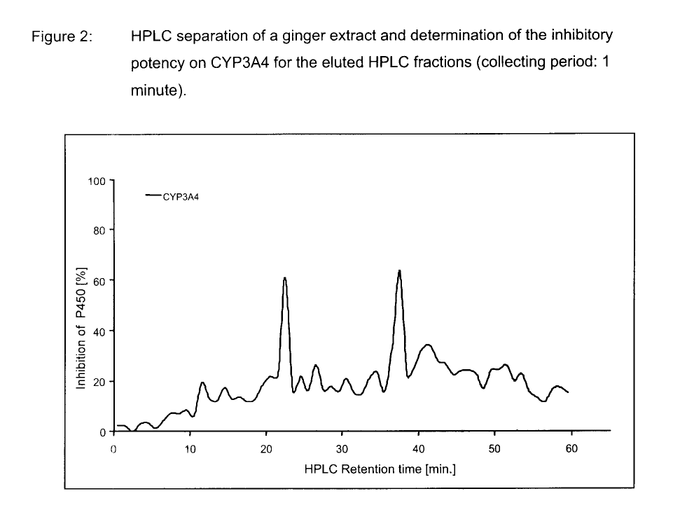

FIG. 3 shows the HPLC separation of a ginger extract and the measurement of the inhibitory potency relative to CYP1A2 for the eluted HPLC fractions (collecting period: 1 minute).

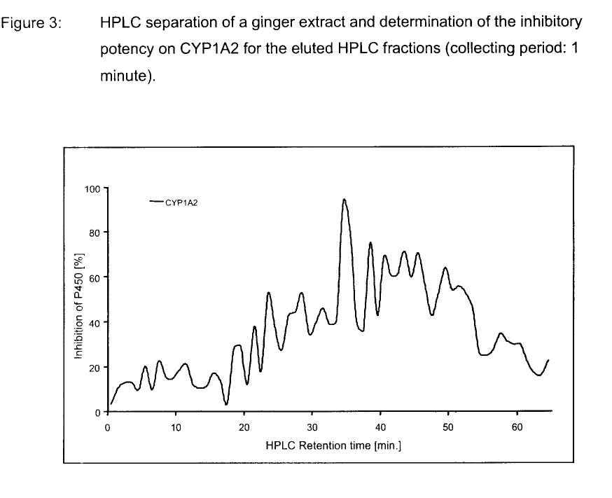

FIG. 4 shows the HPLC separation of a ginger extract and the measurement of the inhibitory potency relative to CYP2C19 for the eluted HPLC fractions (collecting period: 1 minute).

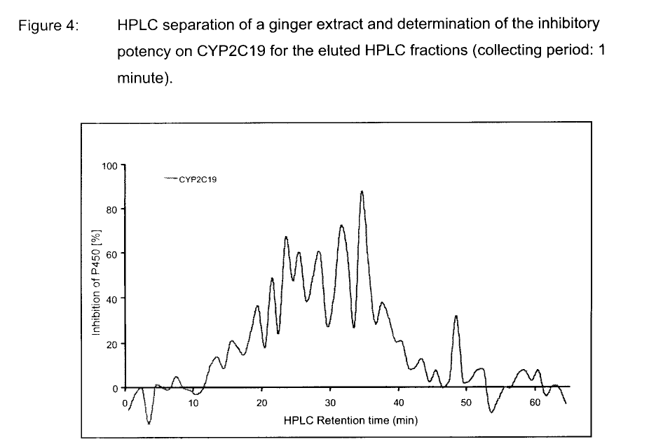

FIG. 5 shows the HPLC separation of a ginger extract and the measurement of the inhibitory potency relative to CYP2C9 for the eluted HPLC fractions (collecting period: 1 minute).

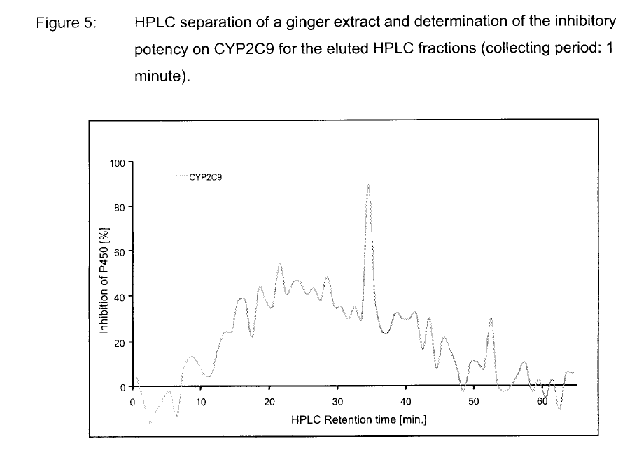

1. A process for isolating a ginger fraction while separating off ethereal oil, comprising the steps of
(a) extracting an oleoresin with a non-polar organic solvent;
(b) extracting the combined residues from step (a) with warm water; and
(c) discarding the supernatant.

2. The process according to claim 1, further characterised in that the combined residues obtained in step (b) are further purified by a process comprising the steps of
(i) extracting with warm alcohol and
(ii) concentrating the combined supernatants from step (i).

3. The process according to claim 1, further characterised in that in step (a) the non-polar organic solvent is a low-boiling alkane solvent, a petrochemical distillate, a propellant or another low-boiling, volatile and non-polar solvent.

4. The process according to claim 1, further characterised in that in step (a) the non-polar organic solvent is hexane.

5. The process according to claim 2, characterised in that in step (i) the warm alcohol is methanol, ethanol, isopropanol, n-propanol, n-butanol or another positionally isomeric butanol, n-pentanol or another positionally isomeric pentanol.

6. The process according to claim 2, characterised in that in step (i) the warm alcohol is methanol.

7. The process according to claim 1, characterised in that the extraction agent used in steps (a) and (b) is used in each case in amounts of 4 to 10 mL/g of the oleoresin used.

8. A ginger fraction obtained by a process according to claim 1.

9. A ginger fraction according to claim 8, characterised in that it contains at least one compound of general formulae 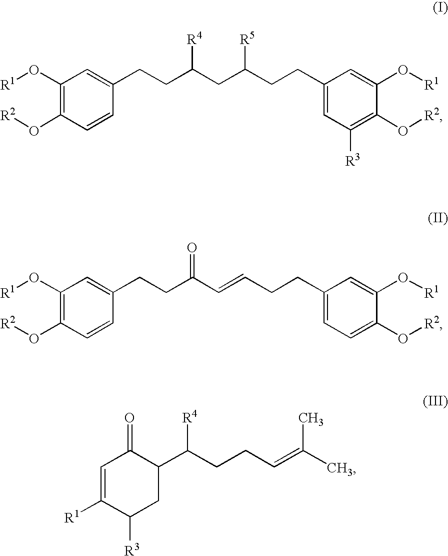
wherein
R¹ denotes H, CH₃,
R² denotes H, CH₃,
R³ denotes H, OH, OCH₃,
R⁴ denotes H, O, OH, OCH₃, OC(O)CH₃ and
R⁵ denotes H, O, OH, OCH₃, OC(O)CH₃,
one of the enantiomers or diastereomers thereof.

10. A ginger fraction according to claim 8, characterised in that it contains at least one of the following compounds: 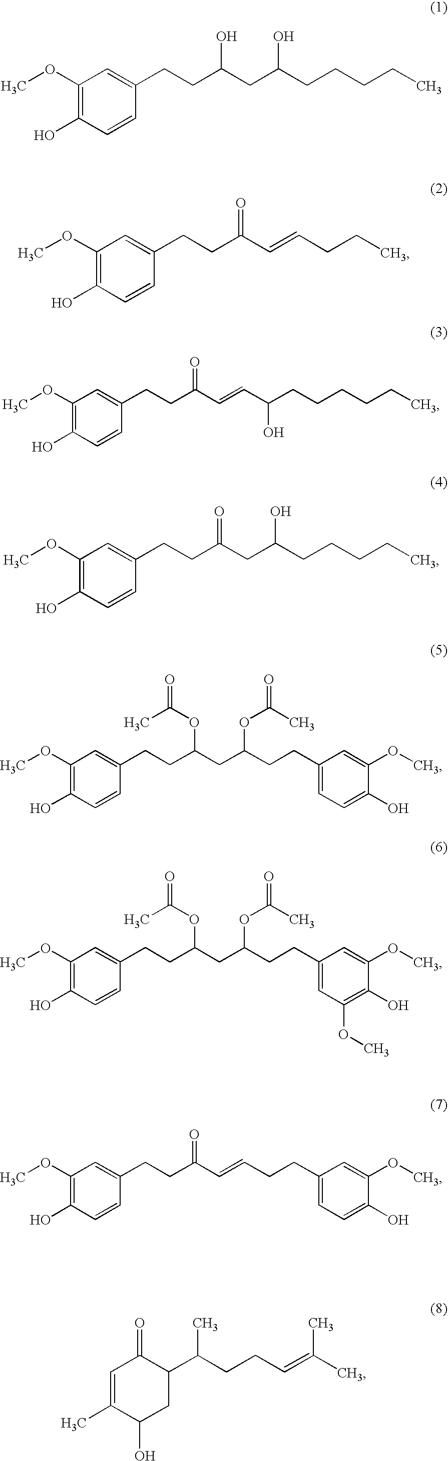
the enantiomers and the diastereomers thereof.

11. A method for inhibiting cytochrome P450 enzymes such as cytochrome P450 3A4 enzymes, cytochrome P450 1A2 enzymes, cytochrome P450 2C9 enzymes and cytochrome P450 2C19 enzymes comprising administration of an effective dose of a ginger fraction of claim 8.

12.-23. (canceled)

24. A pharmaceutical formulation, comprising a ginger function according to claim 9, optionally together with one or more inert carriers and/or diluents.

25. A pharmaceutical formulation, comprising a ginger fraction according to claim 10, optionally together with one or more inert carriers and/or diluents.

26.-28. (canceled)
